(19) United States
(12) Patent Application Publication
(10) Pub. No.: US 2015/0182638 A1
(43) Pub. Date: Jul. 2, 2015

(54) VIRUS-MEDIATED DELIVERY OF BEVACIZUMAB FOR THERAPEUTIC APPLICATIONS

(71) Applicant: Cornell University, Ithaca, NY (US)

(72) Inventors: Ronald G. Crystal, New York, NY (US); Stephen M. Kaminsky, Bronx, NY (US); Donald Joseph D'Amico, New York, NY (US); Julie Boyer, Guilford, CT (US); Szilard Kiss, New York, NY (US)

(21) Appl. No.: 14/645,630

(22) Filed: Mar. 12, 2015

Related U.S. Application Data
(63) Continuation of application No. 13/537,457, filed on Jun. 29, 2012, now abandoned.
(60) Provisional application No. 61/544,210, filed on Oct. 6, 2011.

(51) Int. Cl. A61K 48/00; C12N 15/86; A61K 9/00; C07K 16/22

(52) U.S. Cl. CPC A61K 48/0075; A61K 48/005; C07K 16/22; C12N 15/86; A61K 9/0048; A61K 2039/505

(57) ABSTRACT

The invention provides a method of inhibiting ocular neovascularization in a mammal by administering a composition comprising a bevacizumab-encoding adeno-associated virus (AAV) vector directly to the eye of the mammal.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 4,577 Byte ASCII (Text) file named “710331_ST25.TXT,” created on Jun. 29, 2012.

### BACKGROUND OF THE INVENTION

Pathological ocular neovascularization is the hallmark of age-related macular degeneration (AMD) and diabetic retinopathy (DR), two of the leading causes of blindness in the industrialized world (see, e.g., Elman et al., Ophthalmology, 117: 1064-1077 (2010); and Folk and Stone, N. Engl. J. Med., 363: 1648-1655 (2010)). The prevalence of AMD in the United States is expected to increase to nearly 3 million by 2020, whereas the prevalence of DR is projected to triple to 16 million by 2050 (see, e.g., Friedman et al., Arch. Ophthalmol., 122: 564-572 (2004); and Saaddine et al., Arch. Ophthalmol., 126: 1740-1747 (2008)). Local up-regulation of the expression of vascular endothelial growth factor (VEGF) plays a central role in the pathogenesis of both disorders (see, e.g., Aiello et al., N Engl. JMed, 331: 1480-1487 (1994); and Ferrara et al., Nat. Med, 16: 1107-1111 (2010)).

The clinical use of intravitreal anti-VEGF agents has been shown to slow the progression of vision loss and improve visual acuity in patients with AMD and DR (see, e.g., Avery et al., Ophthalmology, 113: 363-372 (2006); Rosenfeld et al., N Engl. JMed, 355: 1419-1431 (2006); Elman et al., supra; and Gulkilik et al., Int. Ophthalmol., 30: 697-702 (2010)). A widely used anti-VEGF ocular therapy is bevacizumab (AVASTIN™; Genentech, Inc., South San Francisco, Calif.), which is a humanized monoclonal antibody (mAb) specific for human VEGF (see, e.g., Ferrara et al., Nat. Rev. Drug Discov., 3: 391-400 (2004); Avery et al., supra, and U.S. Pat. No. 6,884,879). Numerous clinical studies have established that intravitreal administration of bevacizumab inhibits VEGF-dependent neovascularization and vascular permeability, improves visual outcomes, and decreases vision loss in patients with AMD and DR. Since their introduction, intravitreal injections of bevacizumab and its antigen-binding fragment (Fab) ranibizumab have become the standard of care for treatment of AMD and are becoming the standard of care for DR, especially diabetic macular edema (see, e.g., Avery et al., supra; Gulkilik et al., supra; Nicholson and Schachat, Graefes Arch. Clin. Exp. Ophthalmol., 248: 915-930 (2010); Arevalo et al., J. Ophthalmol., Article ID 584238 (2011); Montero et al., Curr. Diabetes Rev., 7: 176-184 (2011); Ozturk et al., J. Ocul. Pharmacol. Ther., 27: 373-377 (2011); Salam et al., Acta Ophthalmol., 89: 405-411 (2011); and Witkin and Brown, Curr. Opin. Ophthalmol., 22: 185-189 (2011)). However, the positive effect on visual acuity is often of limited duration, with the need for repeated (typically monthly) injections to achieve optimal visual outcome (see, e.g., Regillo et al., Am J Ophthalmol., 145: 239-248 (2008); Elman et al., supra; Gulkilik et al., supra; Mitchell et al., Br. J Ophthalmol., 94: 2-13 (2010); and Schmidt-Erfurth et al., Ophthalmology, 118(5): 831-839 (2011) (Epub Dec. 13, 2010)).

Repeated intravitreal administrations pose significant burdens on the patient and the health care system, and also pose a risk of potentially devastating ocular complications. The most serious adverse event is infectious endophthalmitis. More frequent, although less devastating, adverse events associated with repeated intravitreal administrations include vitreous hemorrhage, retinal detachment, traumatic cataract, corneal abrasion, subconjunctival hemorrhage, and eyelid swelling (see, e.g., Jager et al., Retina, 24: 676-698 (2004); Brown et al., N Engl. J Med, 355: 1432-1444 (2006); Rosenfeld et al., supra; Elman et al., supra; and Folk and Stone, supra)

Thus, there remains a need for improved methods for intraocular delivery of bevacizumab with reduced side effects. The invention provides such methods.

### BRIEF SUMMARY OF THE INVENTION

The invention provides a method of inhibiting ocular neovascularization in a mammal. The method comprises administering a composition comprising an adeno-associated virus (AAV) vector and a pharmaceutically acceptable carrier directly to the eye of a mammal, wherein the AAV vector comprises a nucleic acid sequence encoding bevacizumab, or an antigen-binding fragment thereof, whereupon the nucleic acid sequence is expressed in the eye and ocular neovascularization is inhibited in the mammal.

### BRIEF DESCRIPTION OF THE SEVERAL VIEWS OF THE DRAWING(S)

FIG. 1 is a diagram which schematically depicts the bevacizumab cDNA expression cassette described in Example 1. “CMV” denotes the cytomegalovirus-chicken β-actin promoter.

FIGS. 2A and 2B are images which depict experimental data from a Western blot to assay expression of bevacizumab from the AAV vector denoted AAVrh.10BevMab (see Example 2). FIG. 2A depicts a nonreducing Western analysis (lane 1—supernatant from AAVrh.10BevMab; lane 2—AAVrh.10GFP control; and lane 3—bevacizumab alone). FIG. 2B depicts a reducing Western analysis (lane 4—AAVrh.10BevMab; lane 5—AAVrh.10GFP; lane 6—bevacizumab control). The full-length heavy and light chains of bevacizumab have molecular masses of 50 and 25 kDa, respectively.

FIG. 2C is an image which depicts experimental data from a Western blot to assay the specificity of bevacizumab produced by AAVrh.10BevMab for human VEGF. The left panel depicts results using supernatants from AAVrh.10BevMab-infected cells (lane 7—specificity for mouse VEGF-164; lane 8—specificity for human VEGF-165). The right panel depicts results using supernatants from AAVrh.10GFP-infected cells (lane 9—specificity for mouse VEGF-164; lane 10—specificity for human VEGF-165). Human VEGF-165 has a molecular mass of 19 kDa.

FIG. 2D is a graph which depicts experimental data illustrating the ability of AAVrh.10BevMab to direct persistent expression of bevacizumab in vivo. Shown are bevacizumab levels after systemic administration of the AAVrh.10BevMab vector. AAVrh.10BevMab (10¹¹ gc) was administered to C57BL/6 mice by the intravenous route, and AAVrh.10LacZ (10¹¹ gc) served as a control. Over 24 weeks after vector administration, bevacizumab levels were measured by a human VEGF-specific ELISA. The data are shown as the geometric mean±standard error from n=5 animals per group.

FIG. 3A is a graph which depicts experimental data illustrating bevacizumab expression levels following intravitreal administration of AAVrh.10BevMab to C57BL/6 mice. Over 24 weeks after vector administration, bevacizumab levels in eye homogenate were measured by a human VEGF-specific ELISA. Data (geometric mean±standard error) were obtained from n=6 eyes for the AAVrh.10BevMab group and n=4 eyes for the AAVrh.10αV control group.

FIG. 3B is an image which depicts experimental data from a Western blot that was performed to confirm the expression of bevacizumab in the eye post intravitreal administration of AAVrh.10BevMab. Lane 1 contained a homogenate from AAVrh.10BevMab-treated eyes. Lane 2 contained a homogenate from AAVrh.10αV-treated eyes. Lane 3 contained a homogenate from naive eyes. Lane 4 contained bevacizumab as a control. The heavy and light chains of bevacizumab expressed by the AAV vector have molecular masses of 50 and 25 kDa, respectively.

FIG. 4A-4C are graphs which depict experimental data illustrating AAVrh.10BevMab-mediated suppression of ocular neovascularization quantified by three investigators blinded to the treatment group. FIG. 4A shows data at day 84 post administration of AAVrh.10BevMab. Lines connect data for PBS-injected mice versus AAVrh.10BevMab-injected mice individually. FIG. 4B shows the average data for PBS-injected mice versus AAVrh.10BevMab-injected mice. FIG. 4C shows the percent reduction in total area of neovascularization per retina for mice treated with AAVrh.10BevMab as compared to mice receiving PBS, calculated as indicated in Example 4. A positive percentage represents a reduction in neovascularization.

### DETAILED DESCRIPTION OF THE INVENTION

The invention is predicated, at least in part, on the ability of adeno-associated virus (AAV) vectors to be safely administered intraocularly to humans and to provide persistent expression of a therapeutic transgene (see e.g., Bainbridge et al., N. Engl. J Med, 358: 2231-2239 (2008); Buch et al., Gene Ther., 15: 849-857 (2008); Roy et al., Hum. Gene Ther., 21: 915-927 (2010); Simonelli et al., Mol. Ther., 18: 643-650 (2010); and MacLachlan et al., Mol. Ther., 19: 326-334 (2010)). A single intravitreal administration of an AAV vector expressing bevacizumab desirably results in sustained intraocular expression of bevacizumab at levels sufficient for long-term suppression of ocular neovascularization with minimal adverse events.

The invention provides a method of inhibiting ocular neovascularization in a mammal. The term “ocular neovascularization,” as used herein, refers to any abnormal or inappropriate proliferation of blood vessels from preexisting blood vessels in the eye (also referred to as “ocular angiogenesis”). Ocular neovascularization can occur in any tissue of the eye, and is described in detail in, e.g., Retinal and Choroidal Angiogenesis, John S. Penn (ed.), Springer, Dordrecht, The Netherlands (2008). For example, ocular neovascularization can occur in the choroid. The choroid is a thin, vascular membrane located under the retina. Neovascularization of the choroid can result from a variety of disorders or injuries, including, for example, photocoagulation, anterior ischemic optic neuropathy, Best's disease, choroidal hemangioma, metallic intraocular foreign body, choroidal nonperfusion, choroidal osteomas, choroidal rupture, bacterial endocarditis, choroideremia, chronic retinal detachment, drusen, deposit of metabolic waste material, endogenous Candida endophthalmitis, neovascularization at ora serrata, operating microscope burn, punctate inner choroidopathy, radiation retinopathy, retinal cryoinjury, retinitis pigmentosa, retinochoroidal coloboma, rubella, subretinal fluid drainage, tilted disc syndrome, Taxoplasma retinochoroiditis, and tuberculosis.

Ocular neovascularization also can occur in the cornea, which is a projecting, transparent section of the fibrous tunic, i.e., the outer most layer of the eye. The outermost layer of the cornea contacts the conjunctiva, while the innermost layer comprises the endothelium of the anterior chamber. Neovascularization of the cornea can occur as a result of, for example, ocular injury, surgery, infection, improper wearing of contact lenses, and diseases such as, for example, corneal dystrophies.

Ocular neovascularization can occur in the retina. The retina is a delicate ocular membrane on which images are received. Near the center of the retina is the macula lutea, which is an oval area of retinal tissue where visual sense is most acute. Common causes of retinal neovascularization include ischemia, viral infection, and retinal damage. Retinal neovascularization also can be caused by ocular disorders including, for example, age-related macular degeneration (AMD) and diabetic retinopathy (DR). Neovascularization of the retina can lead to, for example, macular edema, subretinal discoloration, scarring, and hemorrhaging. As a result, vision often is impaired as blood fills the vitreous cavity without being efficiently removed. Not only is the passage of light impeded, but an inflammatory response to the excess blood and metabolites can cause further damage to ocular tissue. In addition, the new vessels form fibrous scar tissue, which, over time, will disturb the retina causing retinal tears and detachment.

In a preferred embodiment, the inventive method inhibits ocular neovascularization associated with age-related macular degeneration (AMD) or diabetic retinopathy (DR). Age-related macular degeneration (AMD) is a progressive, degenerative disorder of the eye that initially causes loss of visual acuity. Advanced age-related macular degeneration occurs in atrophic (or “dry”) and exudative (or “wet”) forms. The “dry” form of advanced AMD, also referred to as “central geographic atrophy,” results from atrophy to the retinal pigment epithelial layer below the retina, which causes vision loss through loss of photoreceptors (rods and cones) in the central part of the eye. The “wet” form of advanced AMD (also referred to as “neovascular” or “exudative” AMD) causes vision loss due to abnormal blood vessel growth (i.e., choroidal neovascularization) in the choriocapillaris, through Bruch's membrane, ultimately leading to blood and protein leakage below the macula. Bleeding, leaking, and scarring from these blood vessels eventually cause irreversible damage to the photoreceptors and rapid vision loss if left untreated. About 10% of patients suffering from AMD have the “wet” form. The clinical features and physiology of AMD are reviewed in, for example, de Jong et al., N. Engl. J. Med., 355: 1474-1485 (2006).

Diabetic retinopathy (DR) is a complication of diabetes that can eventually lead to blindness. DR can occur in Type I or Type II diabetes, and is subdivided into a nonproliferative stage and a proliferative stage. The nonproliferative stage typically develops first, while the proliferative stage is the more advanced and severe form of the disease. Vision loss associated with nonproliferative diabetic retinopathy occurs as a result of retinal edema, in particular diabetic macular edema, which results from vascular leakage. Focal and diffuse vascular leakage occurs as a result of microvascular abnormalities, intraretinal microaneurysms, capillary closure, and retinal hemorrhages. Prolonged periods of vascular leakage ultimately lead to thickening of the basement membrane and formation of soft and hard exudates. Nonproliferative diabetic retinopathy also is characterized by loss of retinal pericytes. The proliferative stage of diabetic retinopathy is characterized by neovascularization and fibrovascular growth (i.e., scarring involving glial and fibrous elements) from the retina or optic nerve over the inner surface of the retina or disc or into the vitreous cavity. The pathology of diabetic retinopathy is described in detail in, e.g., Shah C. A., Indian J. Med. Sci., 62(12): 500-19 (2008), Aiello et al., Diabetes Care, 21: 143-156 (1998), and Watkins, P. J., British Medical Journal, 326(7395): 924-926 (2003).

The inventive method comprises administering a composition comprising an adeno-associated virus (AAV) vector which comprises a nucleic acid sequence encoding bevacizumab, or an antigen-binding fragment thereof. Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61: 447-57 (1990)). These proteins also regulate transcription from endogenous AAV promoters and promoters within helper viruses (see, e.g., Pereira et al., J. Virol., 71: 1079-1088 (1997)). The other Rep proteins modify the function of Rep78 and Rep68. The cap genes encode the capsid proteins VP1, VP2, and VP3. The cap genes are transcribed from the p40 promoter.

The AAV vector used in the inventive method can be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316-327 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-6 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, B. J., Hum. Gene Ther., 16: 541-550 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71: 6823-33 (1997); Srivastava et al., J. Virol., 45: 555-64 (1983); Chiorini et al., J. Virol., 73: 1309-1319 (1999); Rutledge et al., J. Virol., 72: 309-319 (1998); and Wu et al., J. Virol., 74: 8635-47 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J. Virol., 73(2): 939-947 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (i.e., functionally substitute) corresponding sequences from other serotypes during production of AAV particles in mammalian cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 13(1): 1-2 (2006), Gao et al., J. Virol., 78: 6381-6388 (2004), Gao et al., Proc. Natl. Acad. Sci. USA, 99: 11854-11859 (2002), De et al., Mol. Ther., 13: 67-76 (2006), and Gao et al., Mol. Ther., 13: 77-87 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). Preferably, the AAV vector is generated using an AAV that infects a non-human primate pseudotyped with an AAV that infects humans. Examples of such pseudotyped AAV vectors are disclosed in, e.g., Cearley et al., Molecular Therapy, 13: 528-537 (2006). In one embodiment, an AAV vector can be generated which comprises a capsid protein from an AAV that infects rhesus macaques pseudotyped with AAV2 inverted terminal repeats (ITRs). In a particularly preferred embodiment, the AAV vector of the inventive method comprises a capsid protein from AAV10 (also referred to as “AAVrh.10”), which infects rhesus macaques pseudotyped with AAV2 ITRs (see, e.g., Watanabe et al., Gene Ther., 17(8): 1042-1051 (2010); and Mao et al., Hum. Gene Therapy, 22: 1525-1535 (2011)).

The AAV vector of the inventive method comprises a nucleic acid sequence encoding bevacizumab, or an antigen-binding fragment thereof “Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides.

Bevacizumab (AVASTIN™, Genenetch, Inc., South San Francisco, Calif.) is a humanized monoclonal antibody that inhibits vascular endothelial growth factor A (VEGF-A) (Ferrara et al., Nat. Rev. Drug Discov., 3(5): 391-400 (2004); Avery et al., Ophthalmology, 113: 363-372 (2006), and U.S. Pat. No. 6,884,879). Bevacizumab was first approved by the U.S. Food and Drug Administration (FDA) for use in combination with chemotherapy for the treatment of metastatic colon cancer. Bevacizumab has since been approved for the treatment of advanced nonsquamous non-small cell lung cancer (NSCLC), metastatic renal cancer, and glioblastoma (see AVASTIN™ prescribing information).

One of ordinary skill in the art will appreciate that an antibody consists of four polypeptides: two identical copies of a heavy (H) chain polypeptide and two copies of a light (L) chain polypeptide. Each of the heavy chains contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody. The AAV vector of the inventive method can comprise one or more nucleic acid sequences, each of which encodes one or more of the heavy and/or light chain polypeptides of bevacizumab. In this respect, the AAV vector of the inventive method can comprise a single nucleic acid sequence that encodes the two heavy chain polypeptides and the two light chain polypeptides of bevacizumab. Alternatively, the AAV vector of the inventive method can comprise a first nucleic acid sequence that encodes both heavy chain polypeptides of bevacizumab, and a second nucleic acid sequence that encodes both light chain polypeptides of bevacizumab. In yet another embodiment, the AAV vector can comprise a first nucleic acid sequence encoding a first heavy chain polypeptide of bevacizumab, a second nucleic acid sequence encoding a second heavy chain polypeptide of bevacizumab, a third nucleic acid sequence encoding a first light chain polypeptide of bevacizumab, and a fourth nucleic acid sequence encoding a second light chain polypeptide of bevacizumab.

The AAV vector of the inventive method can comprise a nucleic acid sequence encoding full-length heavy and light chain polypeptides of bevacizumab. Nucleic acid sequences encoding the full-length heavy and light chain polypeptides of bevacizumab are known in the art (see, e.g., Watanabe et al., supra, Mao et al., supra, and U.S. Pat. No. 6,884,879) and include, for example, SEQ ID NO: 1 and SEQ ID NO: 2, respectively. The AAV vector of the inventive method can comprise a nucleic acid sequence encoding a whole bevacizumab antibody, such as, for example, SEQ ID NO: 3. In another embodiment, the AAV vector can comprise a nucleic acid sequence that encodes an antigen-binding fragment (also referred to as an “antibody fragment”) of bevacizumab. The term “antigen-binding fragment,” refers to one or more fragments of an antibody that retain the ability to specifically bind to an antigen (e.g., VEGF) (see, generally, Holliger et al., Nat. Biotech., 23(9): 1126-1129 (2005)). Examples of antigen-binding fragments include but are not limited to (i) a Fab fragment, which is a monovalent fragment consisting of the V_{L}, V_{H}, C_{L}, and C_{H}1 domains; (ii) a F(ab′)₂ fragment, which is a bivalent fragment comprising two Fab fragments linked by a disulfide bridge at the hinge region; and (iii) a Fv fragment consisting of the V_{L} and V_{H} domains of a single arm of an antibody. In one embodiment, the AAV vector can comprise a nucleic acid sequence encoding a Fab fragment of bevacizumab. An example of a Fab fragment of bevacizumab is ranibizumab (LUCENTIS™, Genentech, Inc., South San Francisco, Calif.), which is derived from the same parent molecule of bevacizumab. Ranibizumab is approved by the FDA for the treatment of wet age-related macular degeneration. Nucleic acid sequences encoding Fab fragments of bevacizumab are known in the art and are disclosed in, for example, Chen et al., Cancer Research, 57: 4593-4599 (1997), and U.S. Pat. No. 6,884,879.

The nucleic acid sequence encoding bevacizumab, or an antigen-binding fragment thereof, can be generated using methods known in the art. For example, nucleic acid sequences, polypeptides, and proteins can be recombinantly produced using standard recombinant DNA methodology (see, e.g., Sambrook et al., Molecular Cloning: A Laboratory Manual, 3^{rd} ed., Cold Spring Harbor Press, Cold Spring Harbor, N.Y., 2001; and Ausubel et al., Current Protocols in Molecular Biology, Greene Publishing Associates and John Wiley & Sons, NY, 1994). Further, a synthetically produced nucleic acid sequence encoding bevacizumab, or an antigen-binding fragment thereof, can be isolated and/or purified from a source, such as a bacterium, an insect, or a mammal, e.g., a rat, a human, etc. Methods of isolation and purification are well-known in the art. Alternatively, the nucleic acid sequences described herein can be commercially synthesized. In this respect, the nucleic acid sequence can be synthetic, recombinant, isolated, and/or purified.

In addition to the nucleic acid sequence encoding bevacizumab, or an antigen-binding fragment thereof, the AAV vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. Preferably, the nucleic acid sequence encoding bevacizumab, or an antigen-binding fragment thereof, is operably linked to a CMV enhancer/chicken β-actin promoter (see, e.g., Niwa et al., Gene, 108: 193-199 (1991); Daly et al., Proc. Natl. Acad. Sci. U.S.A., 96: 2296-2300 (1999); and Sondhi et al., Mol. Ther., 15: 481-491 (2007)).

The inventive method comprises administering a composition comprising the above-described AAV vector and a pharmaceutically acceptable (e.g. physiologically acceptable) carrier. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

The composition is administered directly to the eye of a mammal, such as, for example, a mouse, a rat, a non-human primate, or a human. Any administration route is appropriate so long as the composition contacts an appropriate ocular cell. The composition can be appropriately formulated and administered in the form of an injection, eye lotion, ointment, implant, and the like. The composition can be administered, for example, topically, intracamerally, subconjunctivally, intraocularly, retrobulbarly, periocularly (e.g., subtenon delivery), subretinally, or suprachoroidally. Topical formulations are well known in the art. Patches, corneal shields (see, e.g., U.S. Pat. No. 5,185,152), ophthalmic solutions (see, e.g., U.S. Pat. No. 5,710,182), and ointments also are known in the art and can be used in the context of the inventive method. The composition also can be administered non-invasively using a needleless injection device, such as the Biojector 2000 Needle-Free Injection Management System™ available from Bioject Medical Technologies Inc. (Tigard, Oreg.).

Alternatively, the composition can be administered using invasive procedures, such as, for instance, intravitreal injection or subretinal injection, optionally preceded by a vitrectomy, or periocular (e.g., subtenon) delivery. The composition can be injected into different compartments of the eye, e.g., the vitreal cavity or anterior chamber. Preferably, the composition is administered intravitreally, most preferably by intravitreal injection.

In a preferred embodiment of the invention, the composition is administered once to the mammal. It is believed that a single administration of the composition will result in persistent expression of bevacizumab in the eye with minimal side effects. However, in certain cases, it may be appropriate to administer the composition multiple times during a therapeutic period and/or employ multiple administration routes, e.g., subretinal and intravitreous, to ensure sufficient exposure of ocular cells to the composition. For example, the composition may be administered directly to the eye of the mammal two or more times (e.g., 2, 3, 4, 5, 6, 6, 8, 9, or 10 or more times) during a therapeutic period.

The composition can contact any suitable ocular cell. Ocular cells associated with age-related macular degeneration include, but are not limited to, cells of neural origin, cells of all layers of the retina, especially retinal pigment epithelial cells, glial cells, and pericytes. Other ocular cells that can be contacted as a result of the inventive method include, for example, endothelial cells, iris epithelial cells, corneal cells, ciliary epithelial cells, Mueller cells, astrocytes, muscle cells surrounding and attached to the eye (e.g., cells of the lateral rectus muscle), fibroblasts (e.g., fibroblasts associated with the episclera), orbital fat cells, cells of the sclera and episclera, connective tissue cells, muscle cells, and cells of the trabecular meshwork. Other cells linked to various ocular-related diseases include, for example, fibroblasts and vascular endothelial cells.

As a result of expression of the nucleic acid sequence encoding bevacizumab, or an antigen-binding fragment thereof, in the eye, ocular neovascularization is inhibited in the mammal. Ocular neovascularization is “inhibited” if the ocular neovascularization is reduced or alleviated in a mammal (e.g., a human). Improvement, alleviation, worsening, regression, or progression of ocular neovascularization may be determined by any objective or subjective measure known in the art. Neovascularization can be measured using any suitable assay known in the art, such as, for example, the mouse ear model of neovascularization, the rat hindlimb ischemia model, the in vivo/in vitro chick chorioallantoic membrane (CAM) assay, and the in vitro cellular (proliferation, migration, tube formation) and organotypic (aortic ring) assays (see, e.g., Auerbach et al., Clin. Chem., 49(1): 32-40 (2003)). The inventive method can achieve partial or complete inhibition of ocular neovascularization.

In one embodiment, the inventive method is used to treat an ocular disease, such as age-related macular degeneration (AMD) or diabetic retinopathy (DR) in a mammal, preferably a human. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the composition comprising the bevacizumab-encoding (or bevacizumab fragment-encoding) AAV vector described herein. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the bevacizumab-encoding (or bevacizumab fragment-encoding) AAV vector to elicit a desired response in the individual. The dose of AAV vector in the composition required to achieve a particular therapeutic effect (i.e., inhibition of ocular neovascularization) typically is administered in units of vector genome copies per cell (gc/cell) or vector genome copies/per kilogram of body weight (gc/kg), and this dose will vary based on several factors including, but not limited to, the administration route of the composition, the level of gene expression required to achieve a therapeutic effect, the specific disease or disorder being treated, any host immune response to the AAV vector, and the stability of bevacizumab in the patient. One of ordinary skill in the art can readily determine an appropriate AAV vector dose range to treat a patient having a particular ocular disease or disorder based on these and other factors that are well known in the art.

Alternatively, the pharmacologic and/or physiologic effect may be prophylactic, i.e., the effect completely or partially prevents age-related macular degeneration (AMD) or diabetic retinopathy (DR) in a mammal, preferably a human. In this respect, the inventive method comprises administering a “prophylactically effective amount” of the composition comprising the bevacizumab-encoding (or bevacizumab fragment-encoding) AAV vector described herein to a human that is predisposed to, or otherwise at risk of developing, AMD or DR. A “prophylactically effective amount” refers to an amount effective, at dosages and for periods of time necessary, to achieve a desired prophylactic result (e.g., prevention of disease onset or prevention of disease flare-ups).

The inventive method may be performed in combination with other existing therapies for age related macular degeneration and diabetic retinopathy. For example, the inventive method can be performed in conjunction with the administration of other anti-angiogenic drugs (e.g., pegaptanib (MACUGEN™—Eyetech, Inc., Cedar Knolls, N.J.) or aflibercept (EYLEA™—Regeneron Pharmaceuticals, Inc., Tarrytown, N.Y.)), photodynamic therapy, laser surgery (i.e., laser photocoagulation), and/or surgical removal of the vitreous gel (i.e., vitrectomy).

The following examples further illustrate the invention but, of course, should not be construed as in any way limiting its scope.

### Example 1

This example demonstrates the generation of an adeno-associated virus (AAV) vector comprising a nucleic acid sequence encoding bevacizumab.

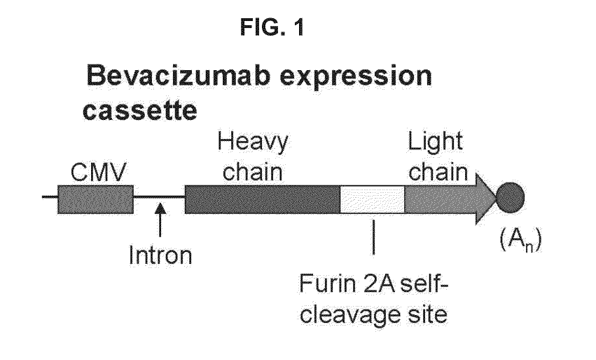

AAVrh.10 is a Glade E, nonhuman primate (rhesus macaque)-derived gene-transfer vector that has been used in human clinical trials for gene therapy for CNS hereditary disease (Sondhi et al., supra). A bevacizumab-encoding AAV vector, AAVrh.10BevMab, was designed based on the AAVrh.10 capsid pseudotyped with AAV2 inverted terminal repeats (ITRs). The ITRs flanked an expression cassette containing (i) the cytomegalovirus (CMV)-enhancer chicken β-actin promoter (Niwa et al., Gene, 108: 193-199 (1991); Daly et al., Proc. Natl. Acad. Sci. U.S.A., 96: 2296-2300 (1999); and Sondhi et al., supra), (ii) nucleic acid sequences encoding the bevacizumab heavy and light chains separated by a furin 2A self cleavage site (Fang et al., Nat. Biotechnol., 23: 584-590 (2005)), and (iii) the rabbit α-globin polyadenylation signal. The bevacizumab cDNA expression cassette is depicted schematically in FIG. 1.

Specifically, nucleotide sequences encoding the bevacizumab heavy and light chain variable domains were derived from the protein sequence for human kappa Fab-12, which is the original humanized version of the murine monoclonal antibody (mAb) corresponding to bevacizumab (Chen et al., J Mol. Biol., 293: 865-881 (1999)). The coding sequences for the human IgG 1 constant domain were added to the variable domain by overlapping PCR.

AAVrh.10BevMab was produced by cotransfection of 293orf6 cells with the following plasmids: (1) an expression cassette plasmid (pAAVrh.10BevMab) (600 pg) comprising cDNA encoding bevacizumab; (2) a packaging plasmid (pAAV44.2) (600 pg) comprising a nucleic acid sequence encoding the AAV2 rep protein and a nucleic acid sequence encoding the AAVrh.10 cap protein (which are necessary for AAV vector replication and capsid production); and (3) pAdDF6 (1.2 mg), an adenovirus helper plasmid (Xiao et al., J Virol., 72: 2224-2232 (1998); and Sondhi et al., supra). 293orf6 cells, which is a human embryonic kidney cell line expressing adenovirus E1 and E4 genes (see, e.g., Gao et al., Proc. Natl. Acad. Sci. U.S.A., 99: 11854-11859 (2002); and Sondhi et al., supra), were cotransfected with the three plasmids using POLYFECT™ (Qiagen, Valencia, Calif.). At 72 hours post-transfection, the cells were harvested, and a crude viral lysate was prepared using four cycles of freeze/thaw and clarified by centrifugation.

AAVrh.10BevMab was purified by iodixanol gradient and QHP anion-exchange chromatography. The purified AAVrh.10BevMab vector was concentrated using an Amicon Ultra-15 100K centrifugal filter device (Millipore, Billerica, Mass.) and stored in PBS, pH 7.4, at −80° C. Using similar methods, three negative control AAV vectors also were prepared: (1) AAVrh.10LacZ, which comprises a nucleic acid sequence encoding β-galactosidase (Wang et al., Cancer Gene Ther., 17: 559-570 (2010)), (2) AAVrh.10GFP, which comprises a nucleic acid sequence encoding green fluorescent protein (GFP) (Sondhi et al., supra), and (3) AAVrh.10αV, which comprises a nucleic acid sequence encoding an unrelated antibody against Y. pestis V antigen. AAV vector genome titers were determined by quantitative TaqMan real-time PCR analysis using a chicken β-actin promoter-specific primer-probe set (Applied Biosystems, Foster City, Calif.).

The results of this example confirm the production of an AAV vector comprising a nucleic acid sequence encoding bevacizumab in accordance with the invention.

### Example 2

This example demonstrates the expression and specificity of bevacizumab expressed from an AAV vector in vitro and in vivo.

The expression and specificity of bevacizumab encoded by AAVrh.10BevMab (described in Example 1) in vitro were assessed using Western blot analysis. For expression analysis, 293orf6 cells were infected with AAVrh.10BevMab (2×10⁵ genome copies (gc)/cell), and infected cell supernatants were harvested 72 hours after infection. Supernatants were concentrated by passage through 1J1tracel YM-10 centrifugal filters (Millipore, Billerica, Mass.) and evaluated by Western analysis using a peroxidase-conjugated goat anti-human kappa light chain antibody (Sigma, St. Louis, Mo.) under nonreducing conditions or reducing conditions with the addition of peroxidase-conjugated goat anti-human IgG antibody (Santa Cruz Biotechnology, Santa Cruz, Calif.). Detection was by enhanced chemiluminescence reagent (GE Healthcare Life Sciences, Piscataway, N.J.).

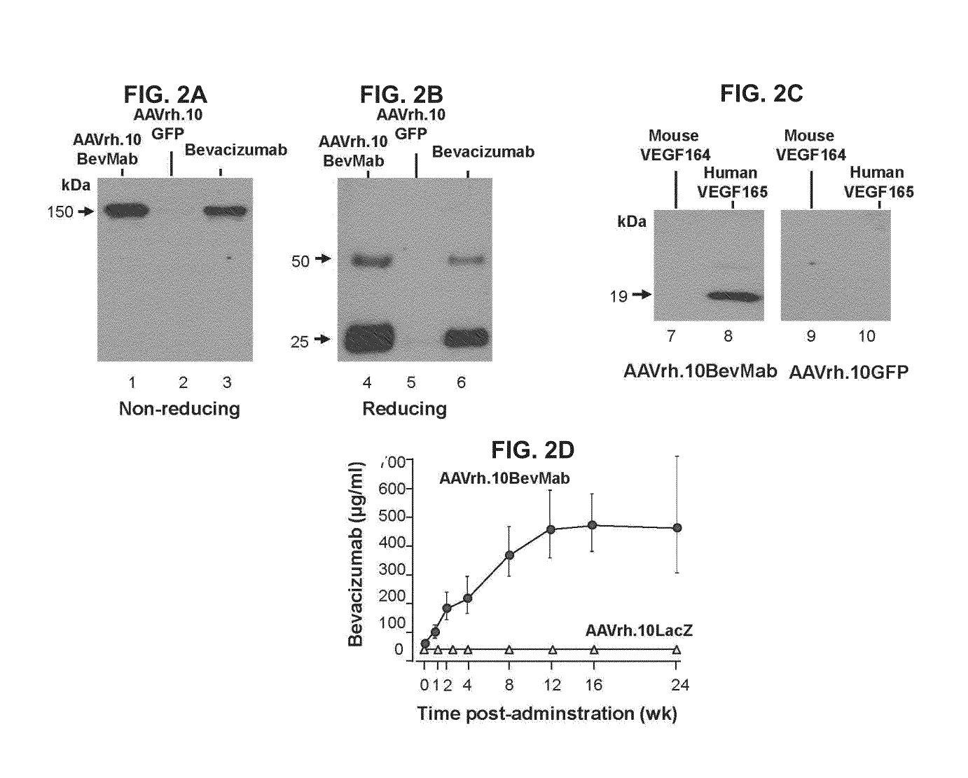

The results of the Western analysis are shown in FIGS. 2A and 2B, and established the expression of the intact heavy and light chains of bevacizumab in 293orf6 cells and their ability to form the intact antibody. Infection with the control AAVrh.10GFP vector under identical reducing or nonreducing conditions gave no detectable bands for human antibody.

Bevacizumab specificity was determined by Western blot analysis against human VEGF-165 and mouse VEGF-164 (Watanabe et al., Hum. Gene Ther., 19: 300-310 (2008)). AAVrh.10BevMab cell supernatants were used as the primary antibody, followed by a peroxidase-conjugated goat anti-human kappa light-chain antibody and enhanced chemiluminescence reagent. The results of the Western analysis are shown in FIG. 2C. Only the human form of VEGF was recognized from the known specificity of bevacizumab. In contrast, supernatants from AAVrh.10GFP-infected cells did not recognize either VEGF protein.

The expression and specificity of bevacizumab in vivo were assessed after administering AAVrh.10BevMab to mice. Specifically, male C57BL/6 mice, 6-8 weeks of age, were obtained from The Jackson Laboratory (Bar Harbor, Me.) and housed under pathogen-free conditions. AAVrh.10BevMab (10¹¹ gc) or negative control AAVrh.10LacZ (10¹¹ gc) in 100 μl of PBS was administered by the intravenous route to C57BL/6 mice through the tail vein.

At various timepoints 0-24 weeks after vector administration, blood was collected through the tail vein, allowed to clot for 60 minutes, and centrifuged at 13,000 rpm for 10 minutes. Bevacizumab levels in serum were assessed by enzyme linked immunosorbent assay (ELISA) using flat-bottomed 96-well EIA/RIA plates (Corning Life Sciences, Lowell, Mass.) coated overnight at 4° C. with 0.2 pg of human VEGF-165 per well in a total volume of 100 μl of 0.05 M carbonate buffer and 0.01% thimerosal. The plates were washed three times with PBS and blocked with 5% dry milk in PBS for 60 minutes. The plates were then washed three times with PBS containing 0.05% Tween 20. Serial serum dilutions in PBS containing 1% dry milk were added to each well and incubated for 60 minutes. The positive control standard was 25 μg/μl bevacizumab (Genentech, Inc., South San Francisco, Calif.). The plates were washed three times with PBS containing 0.05% Tween 20 followed by 100 μl/well 1:5,000 diluted peroxidase-conjugated goat anti-human kappa light chain antibody in PBS containing 1% dry milk for 60 minutes. The plates were then washed four times with PBS containing 0.05% Tween 20 and once with PBS. Peroxidase substrate (100 μl/well; Bio-Rad, Hercules, Calif.) was added, and the reaction was stopped at 15 minutes by addition of 2% oxalic acid (100 μl/well). Absorbance at 415 nm was measured. Antibody titers were calculated using a log (OD)—log (dilution) interpolation model with a cutoff value equal to two-fold the absorbance of background (Watanabe et al., supra). The titers were converted to a bevacizumab concentration using results from the bevacizumab standard data curve.

The results of the ELISA assay are shown in FIG. 2D. Bevacizumab expression levels peaked at about 12 weeks post-administration and were sustained through the 24 week experimental period. Bevacizumab was not detected in the serum of mice that received intravenous injection of the control AAVrh.10LacZ vector.

The results of this example confirm that a nucleic acid sequence encoding bevacizumab is efficiently expressed in vitro and in vivo when delivered via an AAV vector.

### Example 3

This example demonstrates the expression and localization of bevacizumab following intravitreal administration of an AAV vector comprising a nucleic acid sequence encoding bevacizumab.

AAVrh.10BevMab (10¹⁰ gc) (described in Example 1) and control vector AAVrh.10αV (10¹⁰ gc) (described in Example 1) in 1 μl of PBS were administered by intravitreal injection to the left and right eyes, respectively, of C57BL/6 male mice. Intravitreal injection was performed under a dissecting microscope with a 32-gauge needle (Hamilton Company, Reno, Nev.). At various timepoints 0-24 weeks after vector administration, mice were sacrificed with CO₂. Eyes were collected, homogenized by sonication in 100 μl of T-PER tissue protein extraction reagent (Thermo Scientific, Rockford, Ill.), and centrifuged at 13,000 rpm for five minutes, followed by supernatant collection.

Bevacizumab expression levels in the supernatant were assessed by a human VEGF-specific ELISA as described in Example 2. Bevacizumab levels were standardized to total protein levels, which were assayed by a bicinchoninic protein assay (Thermo Scientific, Waltham, Mass.). The expression of bevacizumab in the eye at 12 weeks post-intravitreal injection was evaluated by Western blot analysis as described in Example 2.

To identify the intraocular site of bevacizumab expression, male C57BL/6 mice were injected with AAVrh.10BevMab and AAVrh.10αV, as described above, or were left uninjected. Treated and control virus-injected eyes were enucleated five weeks after intravitreal injection, fixed in formalin, embedded in paraffin wax, sectioned, deparaffinized, and treated sequentially with biotin-conjugated donkey anti-human IgG(H+L) (dilution 1:100; Jackson ImmunoResearch, West Grove, Pa.) and Cy3-conjugated streptavidin (dilution 1:1,000; Jackson ImmunoResearch, West Grove, Pa.). Nuclei were stained with 4′6-diamidino-2-phenylindole (DAPI; dilution 1:2,000; Life Technologies, Carlsbad, Calif.). The sections were embedded (Histoserv, Germantown, Md.) and examined with a fluorescence microscope.

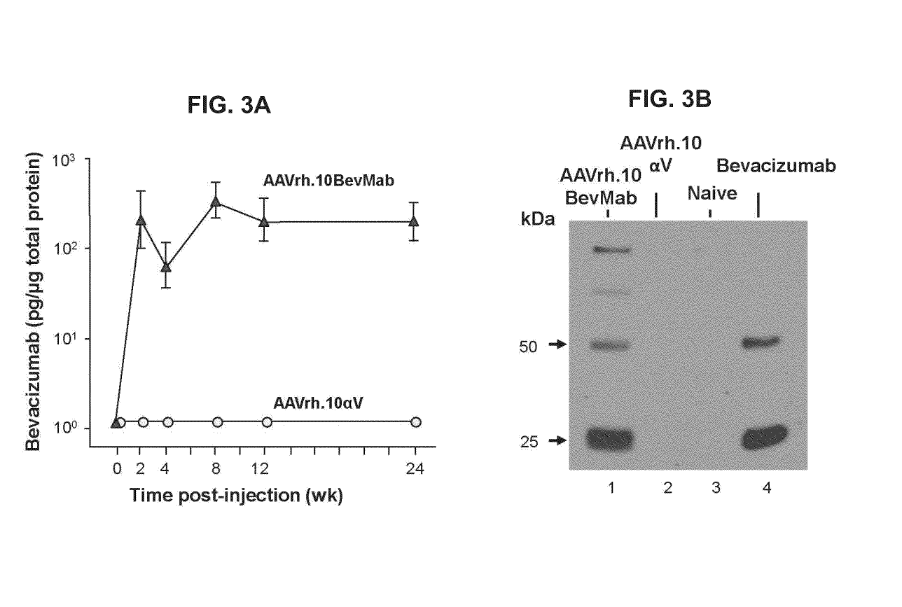

The results of the VEGF-specific ELISA are shown in FIG. 3A. Bevacizumab levels were above 100 pg/pg total protein at two weeks post vector administration and remained at similar levels up to the last time point evaluated at 24 weeks. Bevacizumab was not detected in the eyes from mice that received intravitreal administration of the control vector AAVrh.10αV. The expression of bevacizumab in the eye post-intravitreal administration of AAVrh.10BevMab was confirmed by Western blot analysis, the results of which are shown in FIG. 3B. Soluble protein collected from the AAVrh.10BevMab-injected eyes was positive for the presence of human antibody heavy and light chains, whereas no human antibody was detected in eyes injected with AAVrh.10αV, which expresses a mouse monoclonal antibody, or uninjected naive eyes.

Bevacizumab was localized to the retinal pigment epithelium (RPE), and bevacizumab staining was not observed in uninjected eyes or eyes injected with the control AAVrh.10αV vector. Intravitreal administration of AAVrh.10 has previously been reported to efficiently transduce a wide range of retinal cells, including the RPE, the ganglion cell layer, the amacrine cells of the inner nuclear layer, the Muller and horizontal cells, as well as bipolar cells (see, e.g., Giove et al., Exp. Eye Res., 91: 652-659 (2010)). As such, multiple immunohistochemical sections were searched for staining of these cell types, but no staining was observed in any cell type other than RPE.

The results of this example confirm that bevacizumab is efficiently expressed in mice following intravitreal administration of an AAV vector comprising a nucleic acid sequence encoding bevacizumab, and that bevacizumab expression is localized to retinal pigment epithelium.

### Example 4

This example demonstrates a method of inhibiting ocular neovascularization in a mouse by administering directly to the eye of a mouse a composition comprising a bevacizumab-encoding AAV vector and a carrier.

The efficacy of bevacizumab expressed from the AAVrh.10BevMab vector was assessed in the transgenic rho/VEGF mouse model. Rho/VEGF mice (Okamoto et al., Am J Pathol., 151: 281-291 (1997)) were housed and bred under pathogen-free conditions. At postnatal day 14, homozygous rho/VEGF mice were injected intravitreally with 1 μl of PBS to one eye and 10¹⁰ gc of AAVrh.10BevMab in 1 μl of PBS to the other eye. At 2, 14, 28, 84, and 168 days post-injection, mice were anesthetized and perfused with 2 ml of 25 mg/ml fluorescein-labeled dextran (2×10⁶ average molecular weight; Sigma, St. Louis, Mo.) in PBS. Eyes were removed and fixed for 1 hr in 4% paraformaldehyde/PBS. The cornea and lens were removed, and the entire retina was carefully dissected from the eyecup, radially cut from the edge of the retina to the equator in all four quadrants, and flat-mounted in PROLONG™ Gold antifade reagent (Life Technologies, Carlsbad, Calif.). The retinas were examined by fluorescence microscopy at 200×, providing a narrow depth of field to enable subretinal focus for neovascular buds on the outer surface of the retina. AxioVision LE (Carl Zeiss International, Oberkochen, Germany) digital image analysis software was used by three investigators blinded to treatment group for quantifying subretinal neovascular growth area per retina.

In low-magnification views, multiple large areas of budding and vascular leak were evident in the PBS-treated eye of the mice at 168 days post injection; however, these areas were largely absent in the treated eye. By examining neovascular buds at higher power, the time-dependent increase in budding was observed. At two days post-injection, AAVrh.10BevMab- and PBS-injected eyes appeared to exhibit similar amounts of neovascular buds, but at later time points AAVrh.10BevMab-injected eyes exhibited significantly fewer subretinal neovascular buds than retinas from eyes injected with PBS.

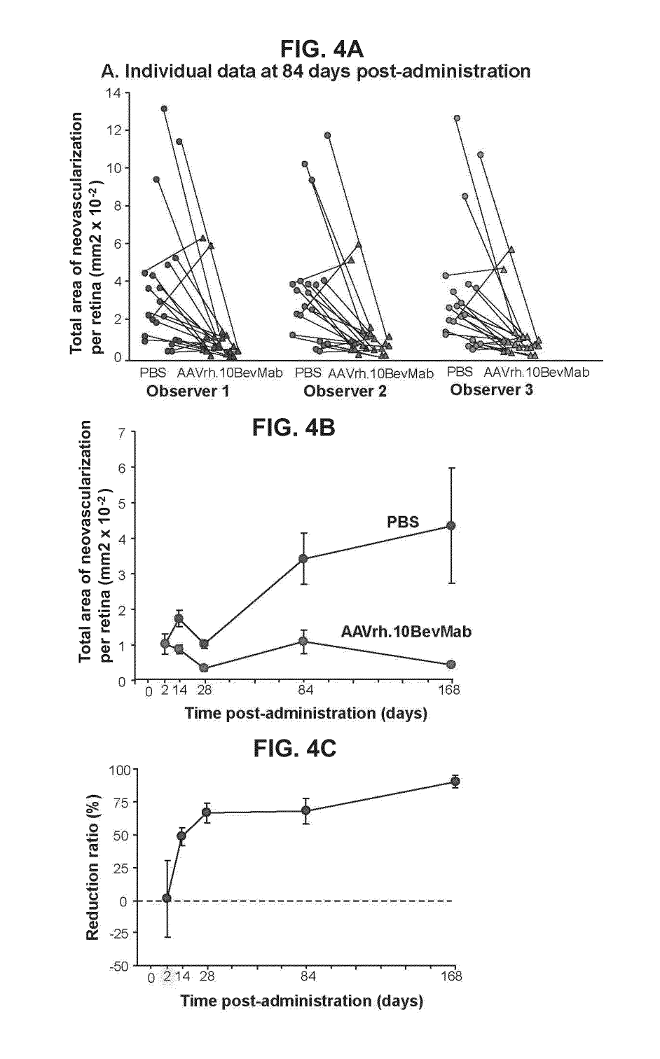

The subretinal neovascular buds were quantified by three investigators blinded to treatment group, and the results of this analysis are shown in FIGS. 4A-4C. As an example of the individual data from each observer, at 84 days post-injection, the data showed a significantly reduced area of subretinal neovascular buds in the retinas of AAVrh.10BevMab-injected eyes compared with eyes injected with PBS (see FIG. 4A). The inter-observer variability in quantifying the neovascular buds was not significant at the multiple test correction threshold, as shown in Table 1.

TABLE 1

| | | Area of NV per retina (mm² × 10⁻²; days) | | | | |
|---|---|---|---|---|---|---|
| Observer | Treatment | 2 | 14 | 28 | 84 | 168 |
| 1 | PBS | 0.90 ± 0.23 | 1.64 ± 0.20 | 1.02 ± 0.11 | 3.61 ± 0.79 | 4.29 ± 1.60 |
| | AAVrh.10BevMab | 0.85 ± 0.22 | 0.81 ± 0.10 | 0.35 ± 0.07 | 1.08 ± 0.37 | 0.43 ± 0.07 |
| 2 | PBS | 1.17 ± 0.30 | 1.98 ± 0.31 | 1.08 ± 0.13 | 3.44 ± 0.70 | 4.49 ± 1.68 |
| | AAVrh.10BevMab | 1.22 ± 0.35 | 1.03 ± 0.16 | 0.33 ± 0.08 | 1.15 ± 0.33 | 0.45 ± 0.06 |
| 3 | PBS | 1.01 ± 0.28 | 1.59 ± 0.21 | 0.94 ± 0.11 | 3.21 ± 0.73 | 4.28 ± 1.60 |
| | AAVrh.10BevMab | 1.00 ± 0.33 | 0.82 ± 0.12 | 0.34 ± 0.09 | 1.15 ± 0.33 | 0.45 ± 0.07 |
| p value | For treatment | >0.96 | <0.0001** | <0.0001** | <0.0001** | <0.0001** |
| (2-way ANOVA) | For observer | >0.52 | >0.243 | >0.76 | >0.92 | >0.99 |
| p value | For treatment | >0.9 | <0.0001** | <0.0001** | <0.0001** | <0.0001** |
| (3-way ANOVA) | For observer | >0.01* | >0.028 | >0.49 | >0.86 | >0.98 |
| | For mouse | <0.0001** | <0.0001** | <0.0001** | <0.0001** | <0.002** |

Observer means and standard deviations were calculated after summing over mice. The effects of treatment, observer, and mouse were assessed using permutations after fitting a two-factor and three-factor ANOVA model.
NV = neovascularization.
*p < 0.05, but not significant after a multiple test correction.
**Significant test results.

Data from the three observers was first averaged for each eye, and then the average and standard error for each condition and time point were plotted (see FIG. 4B). Consistent with the fluorescence microscopy results, at two days post-injection there was no significant reduction in the area of subretinal neovascular buds for AAVrh.10BevMab-injected eyes, but from 14 to 168 days post-injection, eyes injected with AAVrh.10BevMab exhibited a significantly less area of subretinal neovascular buds as compared with retinas from eyes injected with PBS (see FIG. 4B). The reduction ratio was calculated as:

[(mean neovascular bud area in PBS-injected eye at indicated time point)−(neovascular bud area in AAVrh.10BevMab-injected eye at indicated time point)]/(mean neovascular bud area in PBS-injected eye)

The reduction ratio showed no reduction at two days post-injection, but significant reduction was observed at 14 days (49%) to 168 days (90%) post-injection (see FIG. 4C).

The results of this example confirm that a single intravitreal administration of AAVrh.10BevMab can persistently suppress subretinal neovascularization in a mouse in accordance with the inventive method.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting ocular neovascularization in a mammal, which method comprises administering a composition comprising an adeno-associated virus (AAV) vector and a pharmaceutically acceptable carrier directly to the eye of a mammal, wherein the AAV vector comprises a nucleic acid sequence encoding bevacizumab, or an antigen-binding fragment thereof, whereupon the nucleic acid sequence is expressed in the eye and ocular neovascularization is inhibited in the mammal.

2. The method of claim 1, wherein the nucleic acid sequence encodes bevacizumab.

3. The method of claim 2, wherein the nucleic acid sequence encodes an antigen-binding fragment of bevacizumab.

4. The method of claim 1, wherein the mammal is a human.

5. The method of claim 1, wherein the mammal is a mouse.

6. The method of claim 1, wherein the ocular neovascularization is associated with age-related macular degeneration (AMD) or diabetic retinopathy (DR).

7. The method of claim 1, wherein the composition is administered to the mammal intravitreally.

8. The method of claim 1, wherein the composition is administered once to the eye of the mammal.

9. The method of claim 1, wherein the AAV vector is generated using a non-human adeno-associated virus.

10. The method of claim 9, wherein the AAV vector is generated using a rhesus macaque adeno-associated virus.


SEQUENCE LISTING (3 sequences)

SEQ ID NO: 1 - DNA, 369 bp, Artificial Sequence
  Synthetic
gaagtgcagctggtggagtctgggggaggcttggtacagcctggggggtccctgagactctcctgtgcagcctctggatacacctttactaactatggcatgaactgggtccgccaagctccagggaagggcctggagtgggtcggatggattaatacctatacgggagaacctacttatgcagccgattttaaaaggcgattcaccttctctctagacactagcaagagtaccgcgtatctgcaaatgaacagtctgagagctgaggacacggccgtgtattattgtgcaaaatatccccattactacggtagtagtcattggtattttgatgtctggggccagggaaccctggtcaccgtctcctca

SEQ ID NO: 2 - DNA, 321 bp, Artificial Sequence
  Synthetic
gatatccagatgacccagtccccaagctccctgtccgcctctgtgggcgatagggtcaccatcacctgcagcgccagtcaggacatcagcaactatctgaactggtatcaacagaaaccaggaaaagctccgaaagtactgatttactttaccagtagtctccatagtggagtcccttctcgcttctctggatccggttctgggacggatttcactctgaccatcagcagtctgcagccagaagacttcgcaacttattactgtcagcagtacagcacggttccctggacatttggacagggtactaaggtggagatcaaa

SEQ ID NO: 3 - DNA, 2161 bp, Artificial Sequence
  Synthetic
ggtaccaccatggagtttggactgagctgggttttccttgttgctattttaaaaggtgtccagtgtgaagtgcagctggtggagtctgggggaggcttggtacagcctggggggtccctgagactctcctgtgcagcctctggatacacctttactaactatggcatgaactgggtccgccaagctccagggaagggcctggagtgggtcggatggattaatacctatacgggagaacctacttatgcagccgattttaaaaggcgattcaccttctctctagacactagcaagagtaccgcgtatctgcaaatgaacagtctgagagctgaggacacggccgtgtattattgtgcaaaatatccccattactacggtagtagtcattggtattttgatgtctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgagctcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtagaaagaggcgagcacctgtgaaacagactttgaattttgaccttctcaagttggcgggagacgtcgagtccaaccctgggcccgatatccagatgacccagtccccaagctccctgtccgcctctgtgggcgatagggtcaccatcacctgcagcgccagtcaggacatcagcaactatctgaactggtatcaacagaaaccaggaaaagctccgaaagtactgatttactttaccagtagtctccatagtggagtcccttctcgcttctctggatccggttctgggacggatttcactctgaccatcagcagtctgcagccagaagacttcgcaacttattactgtcagcagtacagcacggttccctggacatttggacagggtactaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaaggactagtgg